(19) United States
(12) Patent Application Publication
(10) Pub. No.: US 2021/0290976 A1
(43) Pub. Date: Sep. 23, 2021

(54) PORTAL APPARATUS FOR UV DEACTIVATION OF PATHOGENS

(71) Applicant: Healthe, Inc., Cocoa Beach, FL (US)

(72) Inventors: Fredric Maxik, Cocoa Beach, FL (US); Ran Zhou, Rockledge, FL (US); Addy Widjaja, Palm Bay, FL (US)

(21) Appl. No.: 17/209,418

(22) Filed: Mar. 23, 2021

Related U.S. Application Data
(60) Provisional application No. 62/993,565, filed on Mar. 23, 2020.

(51) Int. Cl. A61N 5/06

(52) U.S. Cl. CPC A61N 5/0624; A61N 2005/0626; A61N 2005/0661; A61N 2005/064

(57) ABSTRACT

A portal device for sanitizing an object is disclosed. The portal device comprises a frame enclosing a portal space sized to permit passage of a human therethrough and one or more irradiating units arranged about the frame. Each irradiating unit comprises one or more ultraviolet (UV) light sources configured to emit UV light to the portal space and one or more sensors configured to detect an object within the portal space. The portal device also comprises a processor configured to receive detection signals from the sensors, activate the UV light sources to emit UV light to the portal space at a wavelength of about 222 nm for a predetermined period of time in response to the detection signals, and deactivate the UV light sources after the predetermined period of time.

### CROSS-REFERENCE TO RELATED APPLICATIONS

The application claims the benefit of priority to U.S. Provisional Application No. 62/993,565 entitled “Portal Apparatus for UV-Deactivation of Pathogens,” filed Mar. 23, 2020, which is incorporated herein by reference in its entirety.

### TECHNICAL FIELD

The present invention relates generally to devices and systems for sanitization of one or more objects within a space and deactivation of pathogens thereon using ultraviolet (UV) light. More particularly, the device may be formed as an entryway and/or may enclose an existing entryway in order to emit UV light to objects within the entryway space. The disclosed devices and systems may be applied to sanitize objects and/or clothing upon entry to a room, building, or other controlled space.

### BACKGROUND

The impact of the spread of viruses has been acutely felt throughout the world in the present time. COVID-19, SARS, and other viruses and microorganisms have had a significant and deadly impact on the way that individuals live their lives. In particular, individuals are less willing and/or able to occupy public spaces, such as malls, restaurants, theaters, public transit states, event and conference spaces, and other crowded locations, for fear of being exposed to and succumbing to a virus.

In order to combat the spread of viruses in public spaces, various precautions have been implemented. Due to the airborne nature of many pathogens including COVID-19, covering one's face with a fabric mask and maintaining physical distance from others is recommended. Proper sanitization of surfaces, especially those that experience frequent human contact, may also be crucial to reduce transmission of pathogens. However, standard cleaning protocols and routines may not efficiently remove pathogens to the degree necessary to significantly impact human-to-human transmission.

More recently, ultraviolet light has been introduced as a means to sanitize surfaces and substances. The type of ultraviolet (UV) light has been classified into at least four bands depending upon the effects upon the skin of humans and other animals. Such bands include UV-A, which is defined as ultraviolet light having a wavelength in a range from 315 nm to 400 nm; UV-B, which is defined as ultraviolet light having a wavelength in a range from 280 nm to 315 nm; UV-C, which is defined as ultraviolet light having a wavelength that is in a range from 235 nm to 280 nm; and Far UV, which is defined as ultraviolet light having a wavelength that is in a range from 185 nm to 235 nm.

Ultraviolet light in the UV-C range has been used for sanitization. For example, UV light emitted at 254 nm and 265 nm has been used to destroy viruses and other microorganisms for a number of years. Far UV light (e.g., 222 nm) has been shown to have some efficacy for this use as well. However, UV light emitted in the UV-C range can have harmful impacts on humans. For example, prolonged direct exposure to UV-C light can result in eye and skin damage, such as acute corneal injury (sometimes referred to as “welder's eye”) and acute erythema. Acute effects from UV-C light include redness, ulceration or burns of the skin. Longer-term effects may include premature aging of the skin and/or skin cancer.

Sanitization of objects and/or clothing may be beneficial upon entry to private or shared public spaces. For example, a room, a building, or another shared space may benefit from implementation of a sanitization procedure as a prerequisite to entry to a space and/or as part of a standard entrance protocol in order to promote or ensure hygiene within the space. While sanitizing with chemicals, wipes, and other cleanings products may be effective, the frequency of ingress and egress of individuals and objects may render such an approach infeasible. Further, although UV sanitization may be suitable for this purpose, a continuously running UV sanitization system may have restrictively large power requirements and/or may be harmful to humans.

As such, it would be desirable to have a sanitization system for entryways that uses UV light and is regulated based on human presence.

### SUMMARY

A portal device for sanitization of objects is provided. The portal device comprises a frame and one or more irradiating units. Each irradiating unit may include one or more ultraviolet (UV) light sources, one or more sensors, a processor, and a non-transitory, computer-readable medium. The frame at least partially encloses a portal space sized and configured to permit passage of a human therethrough. The one or more irradiating units are arranged about the frame. The one or more ultraviolet (UV) light sources are configured to emit UV light to the portal space. The one or more sensors are configured to detect an object within the portal space. The non-transitory, computer-readable medium stores instructions that, when executed, cause the processor to: receive one or more detection signals from the one or more sensors, activate the one or more UV light sources to emit UV light to the portal space at a wavelength of about 222 nm for a predetermined period of time in response to the one or more detection signals, and deactivate the one or more UV light sources after the predetermined period of time.

According to some embodiments, the one or more UV light sources of the one or more irradiating units are configured to emit a sanitizing dose of UV light to the object within the predetermined period of time. In some embodiments, the sanitizing dose is configured to deactivate pathogens on a surface of the object.

According to some embodiments, the frame comprises one or more housing members defining an interior space. In some embodiments, each of the one or more irradiating units are housed within the interior space. According to additional embodiments, the frame further comprises one or more optical members joined with the one or more housing members to enclose the interior space. In some embodiments, the one or more optical members are configured to permit passage of UV light from the one or more UV light sources through the one or more optical members and into the portal space.

According to some embodiments, each irradiating unit further comprises a status indicating device in electrical communication with the processor and configured to emit visible light. In some embodiments, the instructions, when executed, further cause the processor to: control the status indicating device to emit visible light at a first visible wavelength during the predetermined period of time; and control the status indicating device to emit visible light at a second visible wavelength after the predetermined period of time. According to additional embodiments, the instructions, when executed, further cause the processor to: control the status indicating device to emit visible light at a third visible wavelength in response to an error.

According to some embodiments, the one or more sensors comprise passive infrared sensors.

According to some embodiments, the portal device further comprises one or more base members joined to a lower surface of the frame and configured to stabilize the frame in an upright position. According to additional embodiments, the one or more base members are configured to be fixed to a ground surface by one or more fasteners.

An alternate portal device for sanitization of objects is also provided. The portal device comprises a frame and one or more irradiating units. Each of the irradiating units comprise one or more ultraviolet (UV) light sources, one or more sensors, a processor, and a non-transitory, computer-readable medium. The frame at least partially encloses a portal space sized and configured to permit passage of a human therethrough. The one or more irradiating units are arranged about the frame. The one or more ultraviolet (UV) light sources are configured to emit UV light to the portal space. The one or more sensors are configured to detect an object within the portal space. The non-transitory, computer-readable medium stores instructions that, when executed, cause the processor to operate in two modes. In a first mode, the processor controls the one or more UV light sources to continuously emit UV light to the portal space. In a second mode, the processor receives one or more detection signals from the one or more sensors, activates the one or more UV light sources to emit UV light to the portal space at a wavelength of about 222 nm for a predetermined period of time in response to the one or more detection signals, and deactivates the one or more UV light sources after the predetermined period of time.

According to some embodiments, the one or more UV light sources of the one or more irradiating units are configured to emit a sanitizing dose of UV light to the object within the predetermined period of time. In some embodiments, the sanitizing dose is configured to deactivate pathogens on a surface of the object.

According to some embodiments, the frame comprises one or more housing members defining an interior space. In some embodiments, each of the one or more irradiating units are housed within the interior space. According to additional embodiments, the frame further comprises one or more optical members joined with the one or more housing members to enclose the interior space. In some embodiments, the one or more optical members are configured to permit passage of UV light from the one or more UV light sources through the one or more optical members and into the portal space.

According to some embodiments, each irradiating unit further comprises a status indicating device in electrical communication with the processor and configured to emit visible light. In some embodiments, the instructions, when executed, further cause the processor to: in the first mode, control the status indicating device to continuously emit visible light at a first visible wavelength, in the second mode, control the status indicating device to emit visible light at a second visible wavelength during the predetermined period of time; and in the second mode, control the status indicating device to emit visible light at a third visible wavelength after the predetermined period of time. According to additional embodiments, the instructions, when executed, further cause the processor to: control the status indicating device to emit visible light at a fourth visible wavelength in response to an error.

According to some embodiments, the one or more sensors comprise passive infrared sensors.

According to some embodiments, the portal device further comprises one or more base members joined to a lower surface of the frame and configured to stabilize the frame in an upright position. According to additional embodiments, the one or more base members are configured to be fixed to a ground surface by one or more fasteners.

Yet another portal device for sanitization of objects is also provided. The portal device comprises a frame and one or more irradiating units. Each irradiating unit comprises one or more ultraviolet (UV) light sources, one or more sensors, a processor, and a non-transitory, computer-readable medium. The frame at least partially encloses a portal space sized and configured to permit passage of a human therethrough. The one or more irradiating units are arranged about the frame. The one or more ultraviolet (UV) light sources are configured to emit UV light to the portal space. The one or more sensors are configured to detect a presence within the portal space. The non-transitory, computer-readable medium stores instructions that, when executed, cause the processor to: receive one or more presence signals from the one or more sensors indicating the presence within the portal space, and determine, based on the one or more presence signals, whether the presence is a living presence. In response to a determination that the presence is a living presence, the instructions, when executed, cause the processor to activate the one or more UV light sources to emit UV light at a first set of wavelengths to one or more objects in the portal space for a predetermined period of time. In response to a determination that the presence is not a living presence, the instructions, when executed, cause the processor to activate the one or more UV light sources to emit UV light at a second set of wavelengths to the one or more objects in the portal space for the predetermined period of time, and deactivate the one or more UV light sources after the predetermined period of time.

According to some embodiments, the one or more sensors comprise passive infrared sensors configured to detect body heat associated with the living presence.

According to some embodiments, the first set of wavelengths comprises 222 nm.

According to some embodiments, the second set of wavelengths comprises one or more of 254 nm and 265 nm. According to additional embodiments, the second set of wavelengths further comprises 222 nm.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a perspective view of a portal apparatus for utilizing ultraviolet (UV) radiation for the deactivation of pathogens in accordance with an embodiment.

FIG. 2 depicts a perspective view of the portal apparatus of FIG. 1 with a portion of a housing thereof removed in accordance with an embodiment.

FIG. 3 depicts a detailed view of a radiation assembly on the portal apparatus of FIG. 2 in accordance with an embodiment.

FIG. 4 depicts a section view of the radiation assembly of FIG. 3 in accordance with an embodiment.

### DETAILED DESCRIPTION

This disclosure is not limited to the particular systems, devices and methods described, as these may vary. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the devices, systems, and methods can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

The use of the terms first, second, etc. do not denote any order or importance, but rather the terms first, second, etc. are used to distinguish one element from another.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

### Portal Apparatus for UV Deactivation of Pathogens

As discussed herein, it would be advantageous to have a sanitization system for entryways that uses UV light to deactivate pathogens. As generally described herein, the sanitization unit may sanitize objects, clothing, and/or other wearables. In some cases, the sanitization system may be regulated based on the detection of human presence.

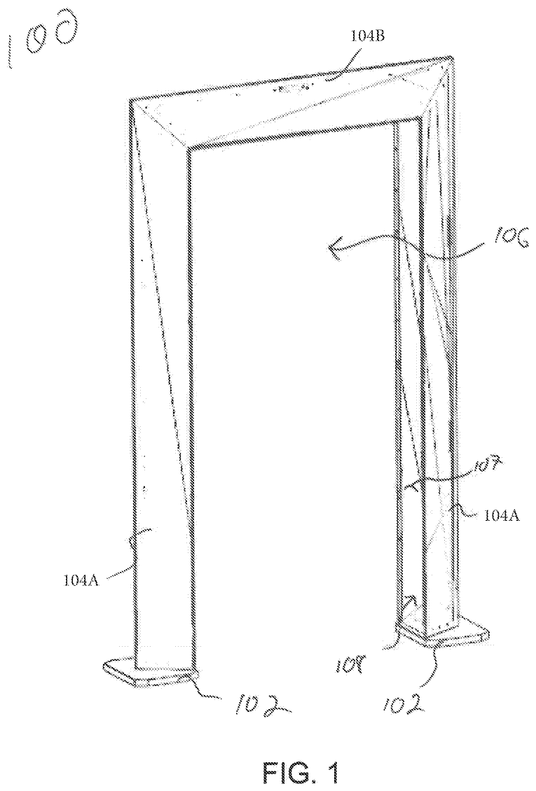

As shown and described by the various figures and accompanying text, embodiments of a portal apparatus that utilize UV radiation to deactivate pathogens within the radiation field of the portal apparatus are provided. Referring now to FIG. 1, a perspective view of the portal apparatus 100 is depicted in accordance with an embodiment. The portal apparatus 100 may comprise base members 102 configured to be positioned on an environmental surface, such as the ground or floor, and one or more housing members 104 attached to the base members 102. The housing members 104 may cooperate to form a frame and to define a portal aperture 106 within the frame. The portal aperture 106 may be the volume of space that is irradiated by the portal apparatus to deactivate pathogens within that space. In some embodiments, the portal aperture 106 may be dimensioned to permit a human to stand within the portal aperture 106 without touching the portal apparatus 100. In the present embodiments, the housing members 104 comprise two vertical housing members 104A parallel to one another and a horizontal housing member 104B attached to upper ends of the vertical housing members 104A. The base members 102 may be attached to lower ends of the vertical housing members 104A and may be flat and have a wide surface area to stabilize the portal apparatus 100 and prevent the portal apparatus 100 from tipping over.

In some embodiments, the housing members 104 define an interior space 108 enclosed therein. The interior space 108 may extend partially or entirely along the housing members 104, i.e., along the periphery of the portal aperture 106 at the sides and the top. In some embodiments, each of the housing members 104 form an opening facing the portal aperture 106.

The portal apparatus 100 may further comprise one or more optical members 107 that are attached to and carried by the housing members 104 to cover the opening of the housing members 104. The optical members 107 may cooperate with the housing members 104 to enclose the interior space 108. The optical members 107 may be configured to permit radiation emitted by the portal apparatus 100 to pass therethrough into the portal aperture 106. For example, the optical members 107 may be formed from polymer, plastic, glass, or another material that is transparent or semi-transparent. In some embodiments, the portal apparatus 100 may not include optical members 107 and the interior space 108 of the housing members 104 may remain open.

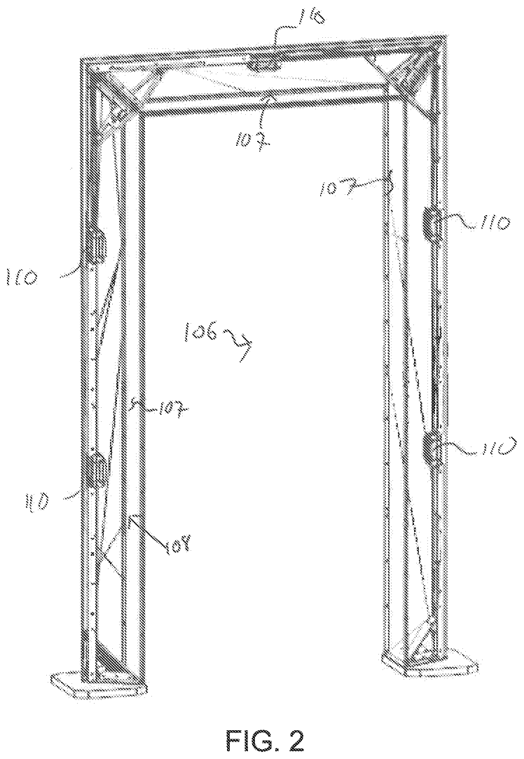

Referring now to FIG. 2, a perspective view of the portal apparatus 100 with a portion of a housing thereof removed is depicted in accordance with an embodiment. The portal apparatus 100 may further comprise one or more radiation assemblies 110. In some embodiments, the portal apparatus 100 comprises a plurality of radiation assemblies 110. The radiation assemblies 110 may be positioned throughout the interior space 108 of the housing members 104, e.g., spaced along the periphery of the portal aperture 106 at the sides and the top. The radiation assemblies may be oriented such that radiation emitted therefrom may pass through the optical members 107 and into the portal aperture 106.

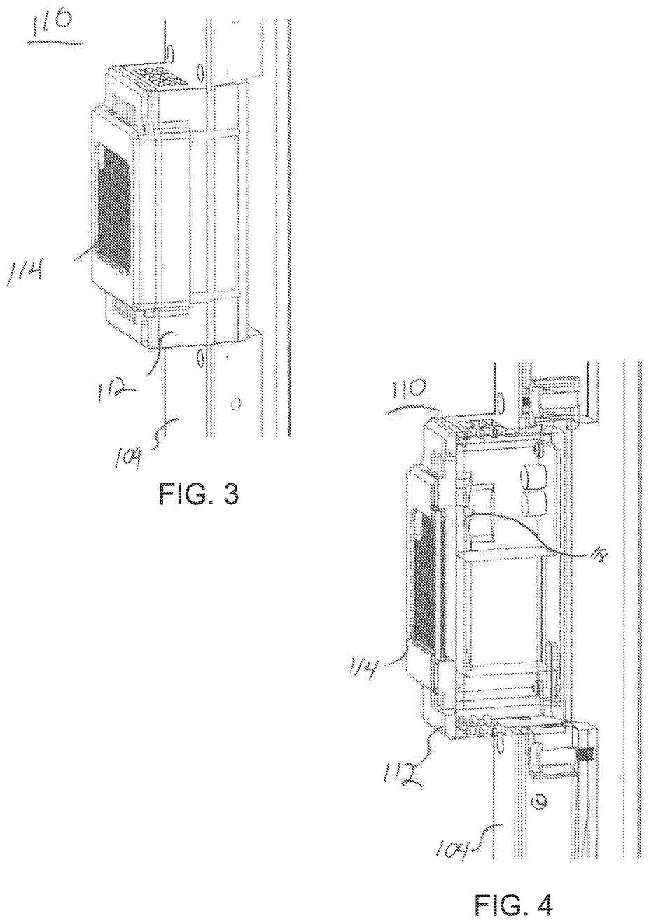

Referring now to FIG. 3, a detailed view of a radiation assembly on the portal apparatus 100 is depicted in accordance with an embodiment. Each radiation assembly 110 may comprise a radiation assembly housing 112 configured to be attached to and carried by the housing member 104, a radiation-emitting device 114 configured to emit radiation through an aperture of the radiation assembly housing 112, and control circuitry 116 in electrical communication with the radiation-emitting device 114 and configured to provide power to and control the operation of the radiation-emitting device 114.

Referring now to FIG. 4, a section view of a radiation assembly on the portal apparatus 100 is depicted in accordance with an embodiment. Each radiation assembly 110 may comprise a sensor 118 positioned in communication with the control circuitry 116 and operable to detect radiation either emitted by or reflected from an object within the portal aperture 106. In some embodiments, the sensor 118 may be configured to detect infrared (IR) radiation resulting from the body heat of an individual occupying the portal aperture 106. In some embodiments, the sensor 118 is a passive infrared (PIR) sensor. In some embodiments, either the sensor 118 or the radiation-emitting device 114 may be operable to emit radiation that can reflect off of objects occupying the portal aperture 106 and be detected by the sensor 118 to indicate the presence of an object in the portal aperture 106. The sensors 118 may additionally or alternatively comprise a variety of types of sensors from which the presence of living specimens (e.g., humans and/or animals) and/or objects may be determined or inferred. For example, the sensors 118 may additionally or alternatively comprise proximity sensors (e.g., ultrasonic proximity sensors, capacitive proximity sensors, infrared proximity sensors, and/or time-of-flight sensors), motion sensors (e.g., cameras or infrared motion sensors), acoustic sensors, and ambient light sensors. In some embodiments, a combination of types of presence sensors as described may be employed in order to more accurately detect the presence of a human, animal, or object. It should be understood that any sensor capable of detecting an object and/or living specimen as would be apparent to a person having an ordinary level of skill in the art is contemplated and included within the scope of the invention.

In some embodiments, the radiation-emitting devices 114 may be removable. For example, where a radiation-emitting device 114 stops functioning or requires repair, the radiation-emitting device 114 may be removed from the housing member 104 and replaced with another radiation-emitting device 114.

As shown in FIG. 2, the portal apparatus 100 may comprise five radiation-emitting devices 114. However, the number of radiation-emitting devices 114 may vary based on the intensity of light emitted by each radiation-emitting devices 114 in order to provide a total sanitizing dose of UV light to objects within the portal aperture 106. Furthermore, the number of radiation-emitting devices 114 may be dependent upon the size of the portal aperture. For example, in some embodiments, the portal aperture 106 forms a volume of about 35.6 in. by about 76.9 in. by about 8.8 in. In such an embodiment, five radiation-emitting devices 114 may be sufficient to provide a sanitizing dose throughout the portal aperture 106. In some embodiments, a larger portal aperture 106 may require more radiation-emitting devices 114. In some embodiments, a smaller portal aperture 106 may require fewer radiation-emitting devices 114.

The control circuitry 116 may be configured to receive signals from the sensor 118 and determine the presence or absence of objects and/or living specimens within the portal aperture 106. Furthermore, the control circuitry 116 may be configured to determine whether an object within the portal aperture 106 is living or non-living. In some embodiments, the control circuitry 116 may interpret IR measurements to determine whether the object is giving off heat consistent with the body of a living specimen.

As described herein, the radiation-emitting device 114 may be configured to emit radiation to deactivate pathogens within the portal aperture 106. Such radiation may be within specific wavelength ranges and have a specific wavelength with a maximum intensity of radiation emitted by the radiation-emitting device. In some embodiments, the radiation-emitting device 114 may be configured to emit electromagnetic radiation having a peak intensity within the Far UV range, i.e. within a range from 180 nm to 235 nm. In some embodiments, the radiation-emitting device 114 may be configured to emit electromagnetic radiation having a peak intensity within a wavelength range from 217 nm to 227 nm. In some embodiments, the radiation-emitting device 114 may be configured to emit electromagnetic radiation having a peak intensity of 222 nm, which is substantially safe for human and/or animal exposure. In some embodiments, the radiation-emitting device 114 may be additionally or alternatively configured to emit electromagnetic radiation having a peak intensity within the UV-C range, i.e. within a range from 235 nm to 280 nm.

It should be understood that the radiation-emitting devices 114 may provide a fluence (e.g., a combined fluence) that effectively sanitizes surfaces of objects within the portal aperture 106 of viruses, bacteria, and/or other pathogens. The total dose of UV radiation to which objects are exposed may be based on the number of radiation-emitting devices 114, the fluence of the radiation-emitting devices 114 upon the objects, and the total exposure time during which UV light is emitted. A predetermined dose (i.e., a sanitizing dose) must be delivered to the surface of each object in order to effectively sanitize the object. In some embodiments, the portal apparatus 100 may be configured to sanitize objects within a specified amount of time. In some embodiments, the portal apparatus 100 may be configured to sanitize objects with 15 seconds, 10 seconds, 5 seconds, 4 seconds, 3 seconds, 2 seconds, 1 second, less than 1 second, or individual values or ranges therebetween. Accordingly, the number of radiation-emitting devices 114, the position and orientation of the radiation-emitting devices 114, and the fluence of the radiation-emitting devices 114 may be selected to provide a sanitizing dose within a selected timeframe.

As mentioned above, the control circuitry 116 may be configured to differentiate between living and non-living specimens within the portal aperture 106. In such embodiments, the radiation-emitting device 114 may further be configured to emit a second electromagnetic radiation in the UV-C range, e.g., having a peak intensity within a range from 249 nm to 259 nm, and in further embodiments to emit electromagnetic having a peak intensity of 254 nm. The radiation-emitting device 114 may further be configured to emit a third electromagnetic radiation having a peak intensity within a range from 260 nm to 270 nm, and in further embodiments to emit electromagnetic having a peak intensity of 265 nm. The control circuitry 116 may be configured to emit the first electromagnetic radiation having a peak intensity at 222 nm when the specimen is determined to be living, and to emit the first, second, and/or third electromagnetic radiations having peak intensities at 222 nm, 254 nm, and/or 265 nm, respectively, upon determining the specimen is non-living.

In embodiments utilizing second and third electromagnetic radiations as described, the sensors 118 may also be configured to detect living specimens beyond the portal aperture 106, i.e., within a predetermined distance of the portal aperture 106 such as 1 foot, 2 feet, 3 feet, greater than 3 feet, or individual values or ranges therebetween. In some embodiments, the sensors 118 may detect presence only within the portal aperture 106 and additional sensors may be included for the purpose of detecting the presence of a living specimen within a predetermined distance of the portal aperture 106. The predetermined distance may comprise a safe distance, i.e., a distance beyond which the UV light emitted by the radiation-emitting device 114 is substantially unharmful. The sensors (e.g., proximity sensors) may be tailored to detect a presence of a living specimen within a danger zone and eliminate or ignore a presence of a living specimen at a greater distance to prevent false detections. For example, a human passing at a sufficient distance from the portal apparatus 100 may not be harmed by the second electromagnetic radiation and/or the third electromagnetic radiation. While specific types of sensors may be particularly advantageous for this purpose, any of the sensors described herein and/or additional types of sensors as would be apparent to a person having an ordinary level of skill in the art may be employed to detect presence beyond the portal aperture 106. Accordingly, the control circuitry 116 may be configured to emit the first electromagnetic radiation having a peak intensity at 222 nm when a living specimen is present within the predetermined distance of the portal apparatus 100 and to emit the first, second, and/or third electromagnetic radiations having peak intensities at 222 nm, 254 nm, and/or 265 nm, respectively, when no living specimens are present within the predetermined distance of the portal apparatus 100.

The radiation-emitting device 114 may comprise any device operable to emit radiation within the above-described electromagnetic radiation ranges, including, but not limited to, light-emitting diodes (LEDs), laser diodes (LDs), mercury vapor discharge devices, and/or excimer lamps. Additional and/or alternate types of radiation-emitting devices 114 may also be used as will be apparent to those of ordinary skill in the related art based on the teachings of this disclosure.

In some embodiments, the radiation assemblies 110 may further comprise a status indicating device. In some embodiments, the status indicating device may be one or more LEDs configured to emit light within the visible spectrum. In some embodiments, the status indicating device may be operable to emit light perceived as red, i.e. within a wavelength range from 625 nm to 740 nm when it is desired for the specimen within the portal aperture 106 to indicate an error or otherwise indicate the specimen should not pass through the portal aperture 106. In some embodiments, the status indicating device may be operable to emit light perceived as green, i.e. within a wavelength range from 500 nm to 565 nm, to indicate clearance for the specimen to proceed out of the portal aperture 106. In some embodiments, the status indicating device may be operable to emit light perceived as yellow, i.e. within a wavelength range from 565 nm to 590 nm, to indicate the portal aperture 106 is currently being irradiated with UV radiation. In some embodiments, the status indicating device does not emit light when no presence is detected within the portal aperture 106. It should be understood that other colors or wavelengths may be utilized for each of the status indications described herein and the indication of each status is not specific to the exemplary colors as described.

In some embodiments, each radiation assembly 110 comprises a status indicating device configured to indicate the status of that individual radiation assembly. Accordingly, the status of each radiation assembly 110 as described may be individually indicated. In such embodiments, malfunctions or other issues may be easily pinpointed to a specific radiation assembly.

In some embodiments, a plurality of radiation assemblies 110 or all radiation assemblies 110 communicate with one or more shared status indicating devices. For example, the control circuity 116 of each radiation assembly may electrically communicate with the shared status indicating devices, either directly or via additional components. Accordingly, the status may be indicated as yellow if at least one radiation assembly is currently emitting UV radiation. Furthermore, the status may be indicated as red if at least one radiation assembly reports an error. Furthermore, the status may be indicated as green only if all radiation assemblies indicate clearance for the specimen to proceed out of the portal aperture 106.

It is further contemplated and included within the scope of the invention that a similar radiation methodology may be employed in other devices. For example, a retrofit lighting device, such as one shown in U.S. Pat. No. 7,824,065, the content of which is incorporated herein by reference except to the extent disclosure therein is inconsistent with the disclosure herein, may be configured to selectively emit UV radiation as described hereinabove. Furthermore, a device situated within or replacing a dome light within a passenger vehicle may similarly be operable to emit UV radiation for deactivation of pathogens within the vehicle.

In some embodiments, the control circuitry 116 comprises at least one processor and any number of additional electrical components to monitor and control the function of the portal apparatus 100. In some embodiments, the processor may receive signals from the sensors 118 and activate or deactivate components of the radiation assemblies 110 based on the signals. In some embodiments, the radiation-emitting devices 114 may be selectively activated to emit electromagnetic radiation as described based on the detection of an object or living specimen. In some embodiments, the processor may control the types of electromagnetic radiation emitted by the radiation-emitting device 114 in response to a signal from the sensors 118 indicating the presence of a living specimen.

In some embodiments, the processor may control the radiation-emitting devices 114 to cease emission of one or more types of electromagnetic radiation in response to a signal from the sensors 118 or additional sensors indicating the presence of a living specimen (e.g., where a living specimen moves within a predetermined distance of the portal apparatus and/or the portal aperture 106 during irradiation).

The processor may be configured to activate the radiation-emitting devices 114 for a set amount of time to provide a sanitizing dose to the entirety of the portal aperture 106, i.e., a sanitizing cycle. As described herein, the length of a sanitizing cycle may be based on the number of radiation-emitting devices 114, the position and orientation of the radiation-emitting devices 114, and the fluence of the radiation-emitting device 114. The processor may be configured accordingly to provide sufficient UV radiation to sanitize objects within the portal aperture 106.

In some embodiments, the portal apparatus 100 may include a power source in electrical communication with the control circuitry 116 of each radiation assembly 110 and any additional electrical components, e.g., additional sensors, status indicating devices, and the like. In some embodiments, the power source may include a battery. An electrical connection may be used to connect the power source to the various components. For example, the electrical connection may comprise a wired connection. In some embodiments, the power source is integrated with the control circuitry 116. In an alternate embodiment, the power source may comprise a cable (not shown) that is integral to the portal apparatus 100 and configured to connect to a remote source of power via a plug or other connector at a remote end of the cable. In some embodiments, the portal apparatus 100 may be compatible with voltages between 85 Volts AC and 264 Volts AC. In some embodiments, the portal apparatus 100 has a power consumption of 60 Watts. However, the voltage and power consumption requirements may vary as would be apparent to a person having an ordinary level of skill in the art.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

In some embodiments, the portal apparatus 100 may be configured to continuously emit light at about 222 nm, which is substantially safe for human and/or animal exposure. While the embodiments disclosed herein describe the use of sensors, the portal apparatus 100 may be controlled by the control circuitry to continuously emit light at 222 nm without regard to the presence of objects or lack thereof as detected by sensors. In such embodiments, the portal apparatus 100 may operate without using the sensors and/or the sensors may be excluded from the particular embodiment.

In some embodiments, the portal apparatus 100 may be operable in a plurality of modes. In a first mode, the portal apparatus 100 may be controlled by the control circuitry to continuously emit light at 222 nm. In a second mode, the portal apparatus 100 may be controlled by the control circuitry to activate and deactivate the radiation-emitting devices 114 based on detection of objects within the portal aperture as described herein. Accordingly, the first mode may continuously emit UV light, and the second mode may selectively emit UV light. The second mode may have lower power requirements and may conserve energy and the usable lifetime of the radiation-emitting device 114 by operating the radiation-emitting devices 114 only when an object is present within the portal aperture.

In some embodiments, the portal apparatus 100 may be controlled by the control circuitry to continuously emit light at about 222 nm, which is substantially safe for human and/or animal exposure. Furthermore, the portal apparatus 100 may be controlled by the control circuitry as described herein to control emission of additional types of electromagnetic radiation, e.g., 254 nm and/or 265 nm UV light, to only be emitted when no living specimen is detected or a non-living specimen is detected.

In some embodiments, the portal apparatus 100 may be constructed as a free-standing structure that is movable. In some embodiments, the portal apparatus 100 may be constructed as a fixed structure, e.g., bolted to the ground through the base members 102.

In some embodiments, entryways may be constructed or manufactured with an integrated portal apparatus. For example, an entryway may be constructed with a portal apparatus 100 as described integrated into the walls and ceiling forming the entryway. In some embodiments, hardware for construction of an entryway may comprise a portal apparatus 100 integrated therewith. For example, a door frame may be formed as a portal apparatus 100.

The portal apparatus 100 as described herein may be useful for sanitizing objects, including personal belongings, clothing, and/or other wearables on a living specimen. In some embodiments, an object or living specimen may remain within the portal aperture 106 for a predetermined period of time in order to receive a sanitizing dose of UV light. In some embodiments, an object or living specimen may be held in one or more poses in order to receive a sanitizing dose of UV light at all surfaces of the object or living specimen. For example, an object may be placed in a first orientation within the portal aperture 106 for a first period of time and then re-positioned to a second orientation for a second period of time to sanitize additional surfaces (e.g., the surface of the object that is in contact with the ground and covered in the first orientation). In another example, a person may stand within the portal aperture 106 with legs spread and arms spread and/or raised for a first period of time to sanitize the person's clothing more completely. In some embodiments, the person may move to a second orientation for a second period of time to sanitize additional surfaces. In some embodiments, the person may slowly rotate 360 degrees within the portal aperture 106 to provide more complete sanitization.

The portal apparatus 100 as described herein may be used in a variety of private spaces, e.g., an entryway to a home, office, or other enclosed space. The portal apparatus 100 may also be used at an entryway to a room or space within a home, office, building, or other enclosed space. In some embodiments, the portal apparatus 100 may be used in controlled spaces requiring a high degree of sanitization and/or sterile conditions. For example, the portal apparatus 100 may be useful in hospitals, laboratories, testing facilities, care facilities, and the like. The portal apparatus may also be useful in high traffic areas, e.g., public spaces or commercial spaces, and for large scale events, e.g., conferences, concerts, or other large gatherings in an enclosed space. Non-limiting examples of public spaces where the portal apparatus 100 may be used include banks, airports, retail spaces, office spaces (e.g., rental or shared office spaces), shared conference or meeting spaces, event spaces, and the like.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A portal device for sanitization of objects, the portal device comprising:
a frame at least partially enclosing a portal space, wherein the portal space is sized and configured to permit passage of a human therethrough; and
one or more irradiating units arranged about the frame, each irradiating unit comprising:
one or more ultraviolet (UV) light sources configured to emit UV light to the portal space,
one or more sensors configured to detect an object within the portal space,
a processor, and
a non-transitory, computer-readable medium storing instructions that, when executed, cause the processor to:
receive one or more detection signals from the one or more sensors,
activate the one or more UV light sources to emit UV light to the portal space at a wavelength of about 222 nm for a predetermined period of time in response to the one or more detection signals, and
deactivate the one or more UV light sources after the predetermined period of time.

2. The portal device of claim 1, wherein the one or more UV light sources of the one or more irradiating units are configured to emit a sanitizing dose of UV light to the object within the predetermined period of time, wherein the sanitizing dose is configured to deactivate pathogens on a surface of the object.

3. The portal device of claim 1, wherein the frame comprises one or more housing members defining an interior space, wherein each of the one or more irradiating units are housed within the interior space.

4. The portal device of claim 3, wherein the frame further comprises one or more optical members joined with the one or more housing members to enclose the interior space, wherein the one or more optical members are configured to permit passage of UV light from the one or more UV light sources through the one or more optical members and into the portal space.

5. The portal device of claim 1, wherein each irradiating unit further comprises a status indicating device in electrical communication with the processor and configured to emit visible light,
wherein the instructions, when executed, further cause the processor to:
control the status indicating device to emit visible light at a first visible wavelength during the predetermined period of time; and
control the status indicating device to emit visible light at a second visible wavelength after the predetermined period of time.

6. The portal device of claim 5, wherein the instructions, when executed, further cause the processor to:
control the status indicating device to emit visible light at a third visible wavelength in response to an error.

7. The portal device of claim 1, wherein the one or more sensors comprise passive infrared sensors.

8. The portal device of claim 1, further comprising one or more base members joined to a lower surface of the frame and configured to stabilize the frame in an upright position.

9. The portal device of claim 8, wherein the one or more base members are configured to be affixed to a ground surface by one or more fasteners.

10. A portal device for sanitization of objects, the portal device comprising:
a frame at least partially enclosing a portal space, wherein the portal space is sized and configured to permit passage of a human therethrough; and
one or more irradiating units arranged about the frame, each irradiating unit comprising:
one or more ultraviolet (UV) light sources configured to emit UV light to the portal space,
one or more sensors configured to detect an object within the portal space,
a processor, and
a non-transitory, computer-readable medium storing instructions that, when executed, cause the processor to:
in a first mode, control the one or more UV light sources to continuously emit UV light to the portal space, and
in a second mode:
receive one or more detection signals from the one or more sensors,
activate the one or more UV light sources to emit UV light to the portal space at a wavelength of about 222 nm for a predetermined period of time in response to the one or more detection signals, and
deactivate the one or more UV light sources after the predetermined period of time.

11. The portal device of claim 10, wherein the one or more UV light sources of the one or more irradiating units are configured to emit a sanitizing dose of UV light to the object within the predetermined period of time, wherein the sanitizing dose is configured to deactivate pathogens on a surface of the object.

12. The portal device of claim 10, wherein the frame comprises one or more housing members defining an interior space, wherein each of the one or more irradiating units are housed within the interior space.

13. The portal device of claim 12, wherein the frame further comprises one or more optical members joined with the one or more housing members to enclose the interior space, wherein the one or more optical members are configured to permit passage of UV light from the one or more UV light sources through the one or more optical members and into the portal space.

14. The portal device of claim 10, wherein each irradiating unit further comprises a status indicating device in electrical communication with the processor and configured to emit visible light,
wherein the instructions, when executed, further cause the processor to:
in the first mode, control the status indicating device to continuously emit visible light at a first visible wavelength,
in the second mode, control the status indicating device to emit visible light at a second visible wavelength during the predetermined period of time; and
in the second mode, control the status indicating device to emit visible light at a third visible wavelength after the predetermined period of time.

15. The portal device of claim 14, wherein the instructions, when executed, further cause the processor to:
control the status indicating device to emit visible light at a fourth visible wavelength in response to an error.

16. The portal device of claim 10, wherein the one or more sensors comprise passive infrared sensors.

17. The portal device of claim 10, further comprising one or more base members joined to a lower surface of the frame and configured to stabilize the frame in an upright position.

18. The portal device of claim 17, wherein the one or more base members are configured to be affixed to a ground surface by one or more fasteners.

19. A portal device for sanitization of objects, the portal device comprising:
a frame at least partially enclosing a portal space, wherein the portal space is sized and configured to permit passage of a human therethrough; and
one or more irradiating units arranged about the frame, each irradiating unit comprising:
one or more ultraviolet (UV) light sources configured to emit UV light to the portal space,
one or more sensors configured to detect a presence within the portal space,
a processor, and
a non-transitory, computer-readable medium storing instructions that, when executed, cause the processor to:
receive one or more presence signals from the one or more sensors indicating the presence within the portal space,
determine, based on the one or more presence signals, whether the presence is a living presence,
in response to a determination that the presence is a living presence, activate the one or more UV light sources to emit UV light at a first set of wavelengths to one or more objects in the portal space for a predetermined period of time,
in response to a determination that the presence is not a living presence, activate the one or more UV light sources to emit UV light at a second set of wavelengths to the one or more objects in the portal space for the predetermined period of time, and
deactivate the one or more UV light sources after the predetermined period of time.

20. The portal device of claim 19, wherein the one or more sensors comprise passive infrared sensors configured to detect body heat associated with the living presence.

21. The portal device of claim 19, wherein the first set of wavelengths comprises 222 nm.

22. The portal device of claim 19, wherein the second set of wavelengths comprises one or more of 254 nm and 265 nm.

23. The portal device of claim 22, wherein the second set of wavelengths further comprises 222 nm.
